(19) United States
(12) Patent Application Publication
(10) Pub. No.: US 2003/0040043 A1
(43) Pub. Date: Feb. 27, 2003

(54) Characterization of a FtsZ binding site and uses thereof

(76) Inventors: Steven Haney, Congers, NY (US); Cynthia Hale, University Heights, OH (US); Piet de Boer, University Heights, OH (US); Elizabeth Glasfeld, Garrison, NY (US)

Correspondence Address: Craig J. Arnold, Amster, Rothstein & Ebenstein, 90 Park Avenue, New York, NY, 10016, US

(21) Appl. No.: 09/908,504

(22) Filed: Jul. 18, 2001

Related U.S. Application Data
(60) Provisional application No. 60/221,775, filed on Jul. 31, 2000.

(51) Int. Cl. C07K 14/705; C07H 21/04; C12P 21/02; C12N 5/06

(52) U.S. Cl. 435/69.1; 530/350; 435/325; 435/320.1; 536/23.5

(57) ABSTRACT

The present invention relates to the ZipA-binding site of FtsZ, as determined using genetic and mutagenesis techniques. In particular, the present invention provides a ZipA-binding site of FtsZ, and a molecule having a ZipA-binding site. Also disclosed are mutant FtsZ proteins. The present invention is further directed to a method for identifying an agent which interacts with FtsZ, as well as an agent that interacts with FtsZ at a binding site. Finally, the present invention discloses a method for identifying an agent that interacts with a molecule having a ZipA-binding site, as well as an agent that interacts with a molecule having a ZipA-binding site.

This application claims the benefit of U.S. Provisional Application No. 60/221,775 filed Jul. 31, 2000.

### FIELD OF THE INVENTION

The present invention relates to a ZipA-binding site of FtsZ. The binding site of the present invention has been characterized at the genetic level through mutagenesis techniques. Characterization of the ZipA-binding site of FtsZ is crucial for the identification and the design of agents that inhibit FtsZ-ZipA interaction. Such inhibitors will be particularly useful as antibiotic agents against Gram-negative bacteria. Accordingly, the present invention also provides methods for using the ZipA-binding site of FtsZ, as disclosed herein, to identify agents which inhibit FtsZ-ZipA interaction, as well as the agents so identified.

### BACKGROUND OF THE INVENTION

Cell division is a well-defined, highly-regulated response to growth and developmental signals in all organisms (31). The study of cell division has integrated the events that occur during the physical process of cell division, and has coordinated these with the events that define the cell cycle. In bacteria, the study of cell division has identified many genes active in the formation and cleavage of the septum, the timing and positioning of which are among the most critical aspects of cell division (24). Currently, the earliest step in septum formation that has been identified is the formation of the Z-ring, a circular polymer of the tubulin-like protein, FtsZ (41). From this ring, the cell-division machinery assembles and acts.

Two proteins that act early in cell division, and directly on the Z-ring, are FtsA and ZipA (Z-ring Interacting Protein A) (12, 13, 21, 23). FtsA shows homology to the Hsp70 family of ATPases, and may function by linking septum formation to peptidoglycan synthesis. During the initial stage of cell division, FtsZ moves from the cytoplasm to the division site, where it assembles into a Z-ring. The resultant structure provides a platform from which to recruit other members of the Z-ring.

Unlike FtsZ itself, which has a widespread phylogenetic distribution and is conserved among most bacterial cells, ZipA is not that highly conserved, and is apparently present in a subset of Gram-negative genomes. Although ZipA is essential in Escherichia coli, it does not appear to be present in all prokaryotes, since it has not been identified in the genomes of Bacillus subtilis and Mycobacterium tuberculosis, among others. This is true of other cell-division genes as well. In fact, the conservation of cell-division genes has formed the basis for a number of suppositions regarding the following: the aspects of cell division which may be grouped; the aspects of cell division which may be by-passed by unique relationships between a bacterial species and its animal hosts; and the evolutionary relationships between bacteria and eukaryotes (6). However, it is also possible that some genes that play a conserved role in cell division may not be recognized by automated homology searches, such as BLAST, and that errors in contig assembly have caused important regions of conserved genes to be missed.

Based on sequence similarity, the majority of FtsZ proteins contain three main regions. A highly-conserved N-terminal region of 320 residues has a two-domain structure, as revealed by X-ray analysis (20), and is sufficient for polymerization (40). This is followed by a variable spacer region and a conserved segment of about ten amino acids at the extreme C-terminus. This C-terminal segment is present in at least 24 organisms in which the FtsZ sequence has been reported.

To date, the precise mechanism of the Z-ring assembly, and how it affects cell-wall invagination, remains unknown. Lin, et al., using two-hybrid experiments and a co-sedimentation assay, reported that the interaction between FtsZ and ZipA is mediated by the C-terminal domains of the two proteins (18). Specifically, Lin, et al. described that only the C-terminal domain of ZipA (residues 176-328) is required for interaction with FtsZ, while a region of 63 residues from the C-terminus of FtsZ is known to be required for ZipA binding. Prior to the present invention, however, it was not known precisely which region at the C-terminus of FtsZ comprises the ZipA-binding site.

### SUMMARY OF THE INVENTION

The present invention relates to the ZipA-binding site of FtsZ, as determined using genetic and mutagenesis techniques. In particular, the present invention provides a ZipA-binding site of FtsZ, and a molecule having a ZipA-binding site. Also disclosed are mutant FtsZ proteins.

The present invention is further directed to a method for identifying an agent which interacts with FtsZ, by contacting FtsZ with a candidate agent and assessing the ability of the candidate agent to bind to FtsZ at a binding site. Additionally, the present invention provides an agent that interacts with FtsZ at a binding site.

The present invention further discloses a method for identifying an agent that interacts with a molecule having a ZipA-binding site, by contacting the molecule with a candidate agent and assessing the ability of the candidate agent to bind to the molecule at a ZipA-binding site. Also provided is an agent that interacts with a molecule having a ZipA-binding site.

Additional objects of the present invention will be apparent in view of the description which follows.

### BRIEF DESCRIPTION OF THE FIGURES

FIG. 1 illustrates that the interaction of FtsZ with ZipA in the yeast two-hybrid system is specific and sensitive to mutations in FtsZ. Diploid strains were constructed by mating EGY48 containing either pLexA or pLexA-ZipA with YM4271 containing pB42, pB42-FtsZ (wild type), or pB42-FtsZ^{mut} (designated as pB42-FtsZ* in the figure). Overnight cultures were spotted onto a microtiter plate containing 100 μl of medium per well. Five-μl spots were applied onto plates using a pin arrayer, as indicated in the figure. Plates were incubated at 30° C. for 4 days.

FIGS. 2A and 2B depict the isolation of intragenic suppressors of the FtsZ^{D373G} mutation. (2A) Mutations in FtsZ were generated by mutagenic PCR amplification of the FtsZ^{D373G} allele as a construct in pGAD424. Primers for the amplification were to sequences about 300 bp away from the MCS of the vector, allowing for cloning by in vivo recombination and expression of clones carrying mutations that restored the interaction of FtsZ with ZipA. (2B) Growth phenotypes of pGADGH-FtsZ plasmids were recovered from the screen. Plasmids were transformed into yeast strain CG1945, as indicated, grown overnight, and spotted onto the indicated plates.

FIG. 3 sets forth a summary of mutations isolated by PCR mutagenesis in the two-hybrid system. The FtsZ C-terminus is shown as both the wild type sequence (top line), and as the mutated sequence with the D-to-G mutation that was encoded by the template DNA (second line). Suppressors isolated from this DNA are indicated in the third line. Residues D373 to P375, which comprise the signature DIP sequence, are underlined.

FIG. 4 illustrates the interaction of intragenic suppressors of the FtSZ^{D373G} mutation with ZipA and FtsA in the two-hybrid system. Yeast diploid strains, resulting from crosses of yeast strain CG+, containing pAS2-1 based plasmids, with yeast strain CG-strains, containing pGAD-424 based plasmids, are indicated in the figure. Diploid strains for testing were grown overnight and spotted, as described in FIG. 1, onto the plates indicated in the figure.

FIG. 5 depicts the interaction of wild type and mutant FtsZ proteins with ZipA in vitro, and determination of the dissociation constants for the binding of ZipA with FtsZ and FtsZ mutants. The interaction was assayed, as described in Example 1 (Material and Methods), and the data were fit by linear regression with an equation for a bimolecular interaction. ●=biotin-FtsZ; □=biotin-FtsZ^{D373G, P375L}; ♦=biotin-FtsZ^{D373S}; and C=biotin-FtsZ^{D373G}.

FIG. 6 sets forth a comparison of the interaction of FtsA and ZipA with alanine-scanning mutations in other conserved residues of the FtsZ C-terminus. Strains were constructed as described in FIG. 4. Controls were performed, as described in FIG. 4, but were omitted from the figure for clarity. Data from β-galactosidase activity is expressed as the fraction of activity for the wild type sequence.

FIG. 7 demonstrates that ZipA and FtsA interact with the C-terminus of FtsZ in the yeast two-hybrid system. Strains were constructed as described in FIG. 4. Controls were performed, as described in FIG. 4, but were omitted from the figure for clarity.

FIG. 8 depicts the complete amino acid and nucleotide sequences of E. coli FtsZ (SEQ ID NOS: 1 and 2, respectively).

### DETAILED DESCRIPTION OF THE INVENTION

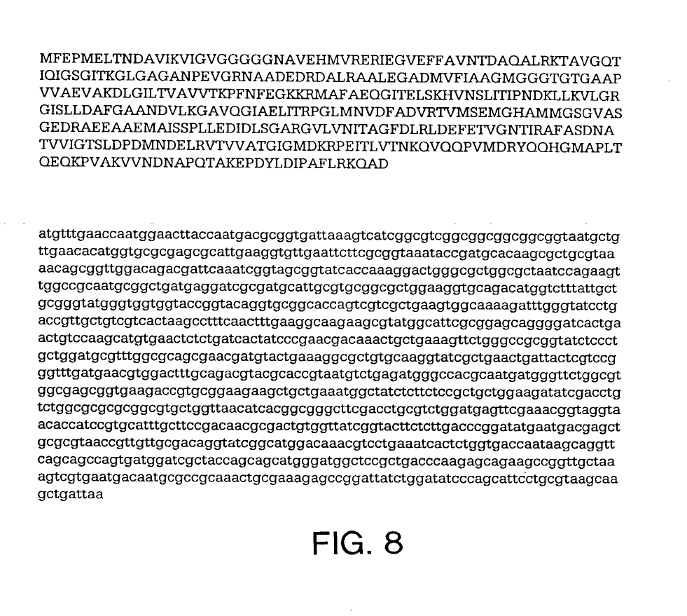

The present invention provides a ZipA-binding site of FtsZ. Unless otherwise indicated, “FtsZ” includes both a “FtsZ peptide” and a “FtsZ analogue”. A “FtsZ peptide” includes at least amino acid residues 367-383 of the C-terminal domain of FtsZ (including conservative substitutions thereof), up to and including a “FtsZ protein” having the amino acid sequence set forth in FIG. 8 (including conservative substitutions thereof). A “FtsZ analogue” is a functional variant of the FtsZ peptide having FtsZ biological activity that is 80% or greater (preferably 90% or greater) in amino-acid-sequence homology with the FtsZ peptide. As further used herein, the term “FtsZ biological activity” refers to the activity of a protein or peptide to interacts with ZipA by binding ZipA at a FtsZ or FtsZ-like binding site, as characterized by the present invention. Preferably, the FtsZ of the present invention is obtained from Escherichia coli. Unless otherwise indicated, “protein” shall include a protein, protein domain, polypeptide, or peptide.

A “binding site” refers to a region of a molecule or molecular complex that, as a result of its shape and charge potential, favorably interacts or associates with another agent—including, without limitation, a protein, polypeptide, peptide, nucleic acid (including DNA or RNA), molecule, compound, antibiotic, or drug—via various covalent and/or non-covalent binding forces. As such, a binding site of the present invention may include the actual site on FtsZ of ZipA binding. A binding site of the present invention may also include accessory binding sites adjacent or proximal to the actual site of ZipA binding that nonetheless may affect FtsZ or FtsZ/ZipA activity upon interaction or association with a particular agent—either by direct interference with the actual site of FtsZ binding, or by indirectly affecting the steric conformation or charge potential of the FtsZ molecule, and thereby preventing or reducing ZipA binding to FtsZ at the actual site of ZipA binding.

“Conservative substitutions” are those amino acid substitutions which are functionally equivalent to the substituted amino acid residue, either because they have similar polarity or steric arrangement, or because they belong to the same class as the substituted residue (e.g., hydrophobic, acidic, or basic). The term “conservative substitutions”, as used herein, includes substitutions having an inconsequential effect on the ability of FtsZ to interact with ZipA at a ZipA-binding site, particularly in respect of the use of said binding site for the identification and design of FtsZ or FtsZ/ZipA complex inhibitors, for molecular replacement analyses, and/or for homology modeling.

The present invention is directed to a ZipA-binding site of FtsZ that, as a result of its shape, reactivity, charge, potential, and other characteristics, favorably interacts or associates with another agent, including, without limitation, a protein (including ZipA), polypeptide, peptide, nucleic acid (including DNA or RNA), molecule, compound, antibiotic, or drug. Accordingly, the present invention is directed to a ZipA-binding site of FtsZ that comprises amino acid residues D370, Y371, L372, I374, F377, L378, and Q381 of FtsZ. These are critical residues in the ZipA-binding site of FtsZ, and may be useful in rational drug design protocols. In a particular embodiment, the ZipA-binding site of FtsZ further comprises amino acid residues D373 and P375 of FtsZ. In another embodiment, the ZipA-binding site of FtsZ further comprises amino acid residues D373, P375, A376, R379, and K380 of FtsZ.

The ZipA-binding site of FtsZ of the present invention may be complexed with a ZipA protein. Accordingly, the present invention further provides a complex comprising the ZipA-binding site of FtsZ of the present invention bound to a C-terminal domain of ZipA. As used herein, the “C-terminal domain of ZipA” means residues 176-328 of ZipA, as well as analogues thereof. In such a FtsZ/ZipA complex, amino acid residues of the ZipA-binding site of FtsZ of the present invention are in direct van der Waal and/or hydrogen bond and/or salt-bridge contact with the amino acid residues of the C-terminal domain of ZipA.

It will be obvious to the skilled practitioner that the numbering of the amino acid residues in the various isoforms of FtsZ or in FtsZ analogues covered by the present invention may be different than that set forth herein, or may contain certain conservative amino acid substitutions that produce the same ZipA-binding activity as that described herein. Corresponding amino acids and conservative substitutions in other isoforms or analogues are easily identified by visually inspecting the relevant amino acid sequences, or by using commercially-available homology software programs.

The present invention is further directed to a molecule having a ZipA-binding site, said molecule consisting of 12 to 30 amino acid residues and said ZipA-binding site including the contiguous peptide sequence DYLDIPAFLRKQ (SEQ ID NO: 3). Unless otherwise indicated, “molecule” shall include a protein, protein domain, polypeptide, or peptide. In a particular embodiment, the molecule of the present invention consists of 12 to 24 amino acid residues. In another embodiment, the molecule of the present invention consists of 12 to 18 amino acid residues. In a further embodiment, the molecule of the present invention is the peptide DYLDIPAFLRKQ (SEQ ID NO: 3). In still another embodiment, the molecule of the present invention is a portion of FtsZ. Preferably, the molecule of the present invention consists of a ZipA-binding site of FtsZ, comprising the contiguous peptide sequence ³⁷⁰DYLDIPAFLRKQ³⁸¹ (SEQ ID NO: 3), including conservative substitutions thereof, as well as amino acid residues both upstream and downstream of the contiguous peptide sequence. The molecule of the present invention may be obtained from bacteria, particularly Gram-negative bacteria. Preferably, the molecule of the present invention is obtained from E coli.

The present invention further provides a mutant FtsZ comprising the amino acid sequence set forth in FIG. 8, in which G is substituted for D at amino acid residue 373. In the method of the present invention, a mutant FtsZ comprising the amino acid sequence set forth in FIG. 8, in which G is substituted for D at amino acid residue 373, may be synthesized by methods commonly known to one skilled in the art (43, 44). Examples of methods that may be employed in the synthesis of the FtsZ amino acid sequence, and a mutant version of this sequence, include, but are not limited to, solid-phase peptide synthesis, solution-method peptide synthesis, and synthesis using any of the commercially-available peptide synthesizers. The mutant FtsZ amino acid sequence of the present invention may contain coupling agents and protecting groups, which are used in the synthesis of protein sequences, and which are well-known to one of skill in the art.

In the method of the present invention, the mutant FtsZ also may be produced from a FtsZ-encoding nucleic acid that has been mutated using methods known to one of skill in the art. These methods of nucleic-acid mutation include, but are not limited to, chemical mutagenesis, disruption (e.g., by allelic exchange), illegitimate recombination, PCR-mediated mutagenesis, signature-tagged mutagenesis, site-directed mutagenesis, targeted gene disruption, and transposon mutagenesis. Preferably, the method of mutation of the present invention is PCR-mediated mutagenesis. The mutated nucleic acid sequence encoding a mutant FtsZ may also be obtained from a library of mutants, wherein mutated bacteria are generated using methods of mutation which include, but are not limited to, chemical mutagenesis, disruption (e.g., by allelic exchange), illegitimate recombination, PCR-mediated mutagenesis, signature-tagged mutagenesis, site-directed mutagenesis, targeted gene disruption, and transposon mutagenesis.

Also provided in the present invention is a mutant FtsZ comprising the amino acid sequence set forth in FIG. 8, in which L is substituted for P at amino acid residue 375. In one embodiment, the mutant FtsZ further comprises G substituted for D at amino acid residue 373. These mutant FtsZ peptides may be generated by any of the above-described methods of amino acid sequence. Moreover, these mutant FtsZ peptides may be produced from FtsZ-encoding nucleic acid sequences that have been mutated by any of the above-described methods of mutagenesis.

Identification of a binding site of a molecule or molecular complex is important because the biological activity of the molecule or molecular complex frequently results from interaction between an agent/ligand and one or more binding sites of the molecule or molecular complex. Therefore, characterization of a binding site of a molecule or molecular complex provides the most suitable tool to be used in identifying inhibitors which affect the activity of the molecule or molecular complex. Characterization of the amino acid sequence of the ZipA-binding site of FtsZ of the present invention also permits the use of various molecular design and analysis techniques for the purpose of designing and synthesizing chemical agents capable of favorably associating or interacting with a ZipA-binding site of FtsZ or a FtsZ analogue, wherein said chemical agents potentially act as inhibitors of FtsZ or FtsZ/ZipA activity.

In view of the foregoing, the ZipA-binding site of FtsZ, as characterized by the present invention, may be used as a tool in the development of drug screens, as a target for small-molecule inhibitors that can act as antibiotics, and as a basis for peptidomimetics. Such drugs, inhibitors, and peptidomimetics may be useful for treating a subject infected with a bacterium, preferably E. coli, by administering to the subject an effective amount of the drug, inhibitor, or peptidomimetic that has been designed in accordance with the method of the present invention. The design and synthesis of an inhibitor of FtsZ biological activity should be relatively simple for two reasons: (1) the ZipA-binding site of FtsZ, as characterized herein, is preferably a small peptide sequence consisting of 12 amino acids; and (2) the substrate of the ZipA-binding site of FtsZ, as characterized in the present invention, is ZipA—a small protein of known molecular structure. FtsZ is a particularly attractive target for rational drug design because this protein, which is commonly found in Gram-negative bacteria, is not found in human cells; therefore, a FtsZ or FtsZ/ZipA inhibitor would not be expected to display toxicity for human cells.

Accordingly, the present invention is directed to a method for identifying an agent which interacts with FtsZ. As used herein, an “agent” shall include a protein, polypeptide, peptide, nucleic acid (including DNA or RNA), antibody, Fab fragment, F(ab′)₂ fragment, molecule, compound, antibiotic, drug, and any combinations thereof. Moreover, an agent which binds to FtsZ may be either natural or synthetic. A Fab fragment is a univalent antigen-binding fragment of an antibody, which is produced by papain digestion. An F(ab′)₂ fragment is a divalent antigen-binding fragment of an antibody, which is produced by pepsin digestion. As used herein, the antibody of the present invention may be polyclonal or monoclonal, and may be produced by techniques well known to those skilled in the art. Polyclonal antibody, for example, may be produced by immunizing a mouse, rabbit, or rat with purified FtsZ. Monoclonal antibody may then be produced by removing the spleen from the immunized mouse, and fusing the spleen cells with myeloma cells to form a hybridoma which, when grown in culture, will produce a monoclonal antibody. The antibody of the present invention also includes a humanized antibody, made in accordance with procedures known in the art.

According to the method of the present invention, an agent which interacts with FtsZ may be identified by contacting FtsZ with a candidate agent, and assessing the ability of the candidate agent to bind to FtsZ at a binding site comprising amino acid residues D370, Y371, L372, I374, F377, L378, and Q381 of FtsZ. In a particular embodiment, the ZipA-binding site of FtsZ further comprises amino acid residues D373 and P375 of FtsZ. In another embodiment, the ZipA-binding site of FtsZ further comprises amino acid residues D373, P375, A376, R379, and K380 of FtsZ.

An agent that binds to FtsZ may be identified using an in vivo assay. For example, comparative binding studies may be performed using a yeast two-hybrid system, whereby interaction between a candidate agent and a wild type FtsZ is compared with interaction between the same candidate agent and a FtsZ which has been mutagenized, according to the methods described above, at one or more of the following amino acid residues: D370, Y371, L372, I374, F377, L378, and Q381. A candidate agent which binds with wild type FtsZ, but which has minimal or no binding with a FtsZ having one or more of the above-described mutations, would be a suitable agent for interacting with FtsZ at a binding site comprising amino acid residues D370, Y371, L372, I374, F377, L378, and Q381 of FtsZ. Similar comparative studies could also be undertaken using in vitro assays, such as an ELISA. Moreover, because the FtsZ substrate, ZipA, is a small protein, a specific inhibitor of FtsZ might be obtained by high-throughput screening of a small-molecule library using purified FtsZ. In one embodiment of the present invention, FtsZ is contacted with the candidate agent in the presence of ZipA. The candidate agent is determined to bind to FtsZ at a binding site comprising amino acid residues D370, Y371, L372, I374, F377, L378, and Q381 of FtsZ by ascertaining if the candidate agent blocks interaction of ZipA with the FtsZ binding site.

The present invention also provides an agent identified by the above-described identification method. Such an agent may be useful for treating a subject infected with a bacterium, particularly E. coli. The subject may be treated by administering to the subject an amount of the agent effective to treat the bacterial infection. The amount of agent required to treat the bacterial infection may be readily determined by one skilled in the art.

The present invention further provides a pharmaceutical composition comprising the agent identified by the above-described identification method and a pharmaceutically-acceptable carrier. The pharmaceutically-acceptable carrier must be “acceptable” in the sense of being compatible with the other ingredients of the composition, and not deleterious to the recipient thereof. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may conveniently be presented in unit dosage. The formulations may be prepared by methods well-known in the pharmaceutical art. For example, the active compound may be brought into association with a carrier or diluent, as a suspension or solution. Optionally, one or more accessory ingredients (e.g., buffers, flavoring agents, surface active agents, and the like) may also be added. The choice of carrier will depend upon the route of administration. The pharmaceutical composition would be useful for administering to a subject an agent which interacts with FtsZ, in order to treat infection with a bacterium, such as E. coli. Where the pharmaceutical composition is administered to a subject to treat a bacterial infection, the agent which interacts with FtsZ is provided in an amount which is effective to treat the bacterial infection in the subject. This amount may be readily determined by the skilled artisan.

The present invention is further directed to a method for identifying an agent which interacts with a molecule having a ZipA-binding site, wherein said molecule consists of 12 to 30 amino acid residues and said ZipA-binding site includes the contiguous peptide sequence DYLDIPAFLRKQ (SEQ ID NO: 3). As described above, the molecule of the present invention may consist of 12 to 24 amino acid residues. In another embodiment, the molecule of the present invention consists of 12 to 18 amino acid residues. In a further embodiment, the molecule of the present invention is the peptide DYLDIPAFLRKQ (SEQ ID NO: 3). In still another embodiment, the molecule of the present invention is a portion of FtsZ. Preferably, the molecule of the present invention consists of a ZipA-binding site of FtsZ, comprising the contiguous peptide sequence ³⁷⁰DYLDIPAFLRKQ³⁸¹ (SEQ ID NO: 3), including conservative substitutions thereof, as well as amino acid residues both upstream and downstream of the contiguous peptide sequence. The molecule of the present invention may be obtained from bacteria, particularly Gram-negative bacteria. Preferably, the molecule of the present invention is obtained from E coli.

In the method of the present invention, an agent which interacts with a molecule having a ZipA-binding site is identified by contacting the molecule with a candidate agent, and assessing the ability of the candidate agent to bind to the molecule at the ZipA-binding site. The agent of the present invention may be identified using an in vivo assay, as described above. For example, comparative binding studies may be performed using a yeast two-hybrid system, whereby interaction between a candidate agent and a molecule having a ZipA-binding site is compared with interaction between the same candidate agent and a molecule having a ZipA-binding site which has been mutagenized, according to the methods described above, at one or more of the amino acid residues of the contiguous peptide sequence DYLDIPAFLRKQ (SEQ ID NO: 3). A candidate agent which binds with the wild type molecule, but which has minimal or no binding with the molecule that has been mutated, would be a suitable agent for interacting with a molecule having a ZipA-binding site and the contiguous peptide sequence DYLDIPAFLRKQ (SEQ ID NO: 3). Similar comparative studies could also be undertaken using in vitro assays, such as an ELISA. Specific inhibitors of the molecule of the present invention also may be obtained by high-throughput screening of a small-molecule library. In one embodiment of the present invention, the molecule of the present invention is contacted with the candidate agent in the presence of ZipA. The candidate agent is determined to bind to the molecule at a binding site having the contiguous peptide sequence DYLDIPAFLRKQ (SEQ ID NO: 3) by ascertaining if the candidate agent blocks interaction of ZipA with the molecule's binding site.

The present invention also provides an agent identified by the above-described identification method. Such an agent may be useful for treating a subject infected with a bacterium, particularly E. coli. The subject may be treated by administering to the subject an amount of the agent effective to treat the bacterial infection. The amount of agent required to treat the bacterial infection may be readily determined by one skilled in the art. Additionally, the present invention further provides a pharmaceutical composition comprising an agent identified by the above-described method and a pharmaceutically-acceptable carrier such as any of the acceptable carriers described above.

Various molecular analysis and rational drug design techniques are further disclosed in U.S. Pat. Nos. 5,834,228, 5,939,528, and 5,865,116, as well as in PCT Application No. PCT/US98/16879 (published as WO 99/09148), the contents of which are hereby incorporated by reference.

The present invention is described in the following example, which is set forth to aid in the understanding of the invention, and should not be construed to limit in any way the scope of the invention as defined in the claims which follow thereafter.

### EXAMPLE 1

1. Introduction

Genetic analyses of cell division have identified many genes as conditional alleles. However, it is difficult to describe an individual protein-protein interaction using detailed genetic analysis, since so many interacting proteins are involved (4, 15, 22). Accordingly, it is advantageous to develop a surrogate system that allows the study of protein-protein interaction at the genetic level. The two-hybrid system of yeast (Y2H) is one such system: there is a wide range of genetic techniques that exist for yeast (1, 11), and the two-hybrid system itself is a robust means of characterizing protein-protein interactions in vivo (7). Thus, it is a relatively simple extension of yeast genetic analysis to examine two-hybrid interaction at the genetic level.

Using the Y2H system, the inventors examined the critical interaction in E. coli cell division between two proteins: FtsZ and ZipA. Previous work had indicated that the C-terminus of FtsZ is important for interaction with both ZipA and FtsA (5, 18, 22). In order to characterize the roles of these interactions, the inventors examined the interaction between FtsZ and ZipA in yeast at the genetic level. They identified mutations in FtsZ specific for the ZipA interaction, characterized the effect of these mutations on the interactions with ZipA and FtsA, and determined the effect of the mutations on cell division.

2. Materials and Methods

Strains and plasmids. All strains and plasmids used in this Example are listed in Table 1, infra, and were prepared as described in more detail below.

Media and reagents. Yeast and bacterial media were prepared by standard methods, using materials readily available (1, 11). YNB, BactoAgar, BactoTryptone, BactoPeptone, and yeast extract were purchased from Difco. Amino acid mixtures (CSM-LUTH and CSM-AHT), raffinose, glucose, and galactose were purchased from Bio101. Amino acids, MUG, and aminotriazole were purchased from Sigma. Zymolyase was purchased from ICN biologicals.

Construction of plasmids. All oligonucleotides used in this Example are listed in Table 2, infra. All plasmids generated by PCR were sequenced on both strands by the Wyeth-Ayerst Research DNA sequencing core facilities in Radnor, Pa. and Pearl River, N.Y. FtsZ and ZipA were cloned by PCR amplification of genomic DNA from E. coli strain MG1655. Wild type FtsZ cloned into pGAD424 was done by PCR amplification of the FtsZ gene from pDR3 using the oligos FtsZ-5′ and FtsZ-3′. The amplified PCR product, which was digested with Mfe I and Sal I, was cloned into pGAD424 that had been digested with EcoRI and BamHI. ZipA was amplified by PCR using oligos ZipA-5′ and ZipA-3′. The ZipA-5′ oligo resulted in a PCR product that deleted the membrane-spanning portion of the ZipA gene product. The resulting fragment was digested with EcoRI and Sal I, and ligated into pLexA to generate plasmid SHp47. This fragment was subsequently excised and cloned into pGAD424 that had been digested identically, to generate plasmid SHp230. The plasmid SHp228 was constructed by PCR amplification of plasmid SHp256 with oligos FtsZ-5′ (short) and FtsZ-3′. The resulting fragment was digested with EcoRI and Sal I, and cloned into pAS2-1. SHp229 was constructed in an identical manner, except that SHp41 was used as the template for the PCR reaction. SHp100 was constructed by PCR amplification of pAS1-FtsA (generously provided by Sandy Silverman) with oligos FtsA-5′ and ADHt. The resulting fragment was digested with EcoRI and Sal I, and ligated into pAS2-1. Plasmid SHp232 was constructed by subcloning the FtsA gene into pGAD424 that had been digested with EcoRI and Sal I as well. pDB312 was prepared by performing a PCR using primers 5′-GGAGGATCCCATATGTTTGAACCAATGGAAC-3′ (SEQ ID NO: 39) and 5′-TTCCGGTCGACTCTTAATCAGCTTGCTTACG-3′ (SEQ ID NO: 40), introducing BamHI, NdeI and SalI sites flanking the ftsz ORF. Digestion with NdeO and SalI resulted in an 1156 bp fragment which was ligated to similarly treated pET21a (Novagen) to yield pDB312. pDR3 was prepared by treating pDB312 with BgIII and HindIII, and ligating the 1268 bp fragment to pMLB1113 (14) which had been treated with BamHI and HindIII.

Construction of yeast strains. Strain SHy9 was generated by growing strain CG1945 serially for two 10-ml overnight cultures, each with an inoculum containing about 10⁵ cells, then plating onto SC plates supplemented with 0.1% FOA. Colonies were allowed to grow for 5 days. Several colonies that grew were reassessed for all phenotypes, including loss of GAL1p-lacZ reporter activity. Strains SHy22 and SHy23 were generated by introducing pHO (kindly provided by Kim Arndt) into strain SHy9, and growing a transformant in 10 ml of SC-URA overnight. Cells were streaked onto a YPD plate, and allowed to grow for 3 days. This plate was then replica-printed onto an SC plate supplemented with 0.1% FOA. Colonies from this plate were patched onto a new YPD plate, grown overnight, and replica-printed onto an SPO plate. This plate was incubated at room temperature for 24 hours, then at 30° C. for 5 days. Patches producing asci were then incubated with Zymolyase. Spores were separated by the random-spores technique, then plated on YPD (33). SHy22 and SHy23 are strains from the same patch, and differ only by mating type.

PCR-mediated mutagenesis and selection of mutations. Mutations in FtsZ were generated by PCR amplification of pGAD424-FtsZ using Taq DNA polymerase and reaction conditions that favored the incorporation of mutations. Oligonucleotides used as primers for amplification were GAL4ad and ADHt. These primers annealed to the GAL4 activation domain and the ADH terminator regions, respectively, and produced a PCR product that included about 300 bp of sequence on either side of the FtsZ gene. These regions of homology allowed for homologous recombination in strain SHy63 of the PCR fragment, when cotransformed with pGADGH vector DNA that had been linearized by digestion with EcoRI and BamHI (29). Recombinants were selected by leucine prototrophy. Cotransformation with 100 ng each of plasmid DNA and PCR fragment resulted in about 1000 colonies, whereas transformation by either the vector or the PCR fragment alone resulted in zero to four colonies when plated onto LT plates.

The interactions between the mutagenized FtsZ gene and ZipA were tested by replica-printing the transformants onto LHT plates supplemented with 0.5 mM AT. Colonies that grew after three days of incubation at 37° C. were scored as hits. Each colony was grown as a 5-ml culture, and miniprepped using the Qiagen Qiaprep Turbo 8 miniprep kit (as described by the manufacturer), except that 0.5 mg of Zymolyase were added per ml of Buffer P1, and samples were incubated for 30 minutes at 30° C. These minipreps were used to transform E. coli strain KC8 by electroporation. Colonies were selected by growth on M9 plates which were supplemented with 50 mg/l ampicillin, but which lacked leucine, as described by Golemis et al. [9]. Two to four colonies from each transformation plate were grown and miniprepped by the Qiagen miniprep kit again, this time without modification to the manufacturer's instructions. DNA samples from these minipreps were analyzed by restriction analysis; the phenotypes were confirmed by transforming into SHy63 again, and rescoring AT resistance. Plasmids were also transformed into a control strain, SHy22, containing the pAS2-1 vector, and phenotypes were checked in these strains as well.

Site-directed mutagenesis of plasmids for expression in yeast and bacteria. Mutations identified above were further characterized by introducing the identified mutation into a pGAD424-FtsZ construct. This was accomplished by site-directed mutagenesis using the Quick Change site-directed mutagenesis kit by Stratagene. Mutagenesis was performed as described by the manufacturer. Oligonucleotides used were: D373G/T, D373G/B; D373S/T, D373S/B; D373G, P375L/T; and D373G, P375L/B. For the alanine-scanning mutations, site-directed changes were introduced into pGAD-FtsZ^{D373G} (SHp201). The mutation that resulted in a change from D to G at position 373 also resulted in a loss of the EcoRV restriction site. Each of the oligo pairs encoded a change that restored this restriction site, in addition to the mutation at the codon to be changed to alanine. The oligos used were: D370A/T, D370A/B; Y371A/T, Y371A/B; L372A/T, L372A/B; F377A/T, F377A/B; L378A/, L378A/B; R379A/T, R379A/B; K380A/T, K380A/B; and Q381A/T, Q381A/B. Candidate clones were screened for the reacquisition of the EcoRV site.

Purification of FtsZ and ZipA, and assay of the FtsZ-ZipA interaction in vitro. The wild type and mutant FtsZ proteins were expressed with the N-terminal biotin tag MAGGLNDIFEAQKIEWH (SEQ ID NO: 38) (34) in order to enable detection in an ELISA. The lysine in this sequence was biotinylated in vivo by the E. coli enzyme, BirA.

Plasmids were constructed by inserting the coding sequence for the biotin tag between the Nco I and Nde I sites of pET28 (Novagen) using the oligos BIOTAG/T and BIOTAG/B. In addition, birA was PCR-amplified from the plasmid pBIOTRX-BirA (38) using the oligos BirA 5′ and BirA 3′, digested with HinD III and Xho I, and ligated into the HinD III and Xho I sites of the same vector into which the biotin tag was inserted, to give the vector pETbio-birA. The genes ftsZ, ftsZ^{D373G}, ftsZ^{D373S}, and fsZ^{D373G, P375L} were subcloned from the vectors pDB312, pEG028, SHp187, and SHp189, respectively, into the Nde I and HinD III sites of pETbio-birA, to give plasmids pEG045, pEG051, pEG052, and pEG053.

Biotin-FtsZ and its mutants were expressed in the E. coli strain BL21(DE3)pLysS. Expression was induced with 1 mM isopropyl-β-D-thiogalacto-pyranoside (IPTG) once the OD₆₀₀ of the culture reached between 0.5 and 1.0. At the same time, D-biotin was added to a final concentration of 0.1 mM. Cells were incubated at 37° C. for another 2-3 hr, centrifuged, and resuspended in buffer A (50 mM Tris, pH 7.9; 50 mM KCl; 1 mM EDTA; and 10% glycerol) and stored at −70° C. The proteins were then purified in accordance with reported procedure (30).

Protein concentrations of biotin-FtsZ and its mutants were determined by the Bradford method. The extent of incorporation of biotin was determined by measuring the displacement of 2-(4′-hydroxyazobenzene) benzoic acid (HABA) from avidin. In short, 40 μl of protein sample or buffer were mixed with 360 μl of 0.5 mg/ml avidin and 0.3 mM HABA in 100 mM sodium phosphate and 150 mM NaCl, pH 7.2. The decrease in absorbance at 500 nM was measured, and the concentration of biotin was determined using Δε₅₀₀×10⁻³=34. The biotin-tagged FtsZ proteins were between 50 and 75% biotinylated.

ZipA(23-328) was overexpressed from the plasmid pDB348 in BL21(DE3)plysS. Expression was induced, as for biotin-FtsZ above, and the cells were similarly centrifuged, resuspended, and stored. At the time of purification, the cells were thawed, phenylmethylsulfonyl fluoride was added, to a concentration of 1 mM, and the cells were lysed by passage through a French press. The cell extract was clarified by centrifugation at 100,000×g for 1 hr, and ZipA(23-328) was precipitated by adding ammonium sulfate to 35% saturation. The ammonium sulfate pellet was dissolved in buffer A and dialyzed against buffer A overnight. ZipA(23-328) was purified to homogeneity by passage over a MonoQ column (Amersham Pharmacia) and elution with a 50-230 mM gradient of KCl in buffer A. The protein concentration of ZipA(23-328) was then determined (8).

The interaction between ZipA(23-328) and biotin-FtsZ and its mutants was assayed in an ELISA format. ZipA(23-328) was immobilized in the wells of an Immulon 4HBX 96-well plate, in 50 mM Tris, pH 8.5 and 100 mM NaCl at 1 μg/ml, overnight at 4° C. Unbound ZipA(23-328) was removed, and the wells were blocked with blocking buffer (0.2% BSA in PBS-T {10 mM Na₂HPO₄; 1.8 mM KH₂PO₄, pH 7.5; 140 mM NaCl; 2.7 mM KCl; and 0.05% Tween 20}). After two washes with PBS-T, biotin-FtsZ and its mutants were added at various concentrations in blocking buffer for 1 hr at room temperature. Unbound FtsZ was removed, and the wells were washed three times with PBS-T. Next, 0.1 μg/ml streptavidin-horse radish peroxidase conjugate in blocking buffer was added, and the wells were then incubated at room temperature for 1 hr. The wells were washed four times after the removal of the conjugate. The horse radish peroxidase substrate o-phenylenediamine was then added in sodium phosphate-citric acid buffer, color development was stopped after a few minutes with 1.3 M H₂SO₄, and the absorbance at 490 nm was measured.

Cell biology methods. Escherichia coli morphology was determined by phase contrast microscopy of E. coli cells, as described previously (13). Experimental conditions are described in the legends for Table 3 and Table 4, infra.

β-Galactosidase assay. Overnight cultures (5 ml) of yeast strains to be assayed for LacZ activity were aliquoted into a 96-well microtiter plate (100 μl per well). Samples were mixed with 100 μl of lysis buffer and substrate (40 μl of Promega Cell Lysis Buffer, 40 μl of 0.125 mg/ml MUG (Sigma), and 20 μl of 10×β-galactosidase assay salts (26)). Cultures were measured in quadruplicate. Samples were incubated at 30° C. for 4-8 hr with shaking, then read on a Victor II fluorescence plate reader from Wallac. Fluorescence intensity increased with time. After 8 hr, negative-control wells showed about 400 units, whereas positive control wells showed about 50,000 units.

TABLE 1
Plasmids, Bacteria, and Yeast used in Example 1
Plasmids

| Name | Common Name | Genotype | Source |
|---|---|---|---|
| SHp40 | pLexA | pBRori 2μ bla HIS3 ADHp-lexA | Clontech |
| SHp47 | | pLexA-ZipA(23-328) | Example 1 |
| SHp42 | pLexA(OP)-8 | pBRori 2μ bla URA3 | Clontech |
| | | GAL1p-lexA(OP)8-lacZ | |
| SHp41 | pB42 | pBRori 2μ bla TRP1 GAL1-B42 | Clontech |
| SHp256 | | pB42-FtsZ | Example 1 |
| SHp48 | | pB42-FtsZ^{D39N,D373G} | Example 1 |
| SHp19 | pAS2-1 | pBRori 2μ bla TRP1 | Clontech |
| | | ADHp-GAL4bd | |
| SHp227 | | pAS2-1-ZipA | Example 1 |
| SHp228 | | pAS2-1-FtsZ(311-383)^{WT} | Example 1 |
| SHp229 | | pAS2-1-FtsZ(311-383)^{D373G} | Example 1 |
| SHp17 | pGAD424 | pBRori 2μ bla LEU2 | Clontech |
| | | ADHp-GAL4ad | |
| SHp232 | | pGAD424-FtsA | Example 1 |
| SHp236 | | pGAD424-ZipA | Example 1 |
| SHp27 | | pGAD424-FtsZ^{D39N,D373G t} | Example 1 |
| SHp261 | pGADGH | pBRori 2μ bla LEU2 | Clontech |
| | | ADH1p-GAL4ad | |
| SHp262 | | pGADGH-FtsZ^{D39N} | Example 1 |
| SHp263 | | pGADGH-FtsZ^{D39N} | Example 1 |
| SHp264 | | pGADGH-FtsZ^{D39N,D373S} | Example 1 |
| SHp265 | | pGADGH-FtsZ^{D39N,D373S} | Example 1 |
| SHp266 | | pGADGH-FtsZ^{D39N,D373C} | Example 1 |
| SHp267 | | pGADGH-FtsZ^{D39N,D373G,P375L} | Example 1 |
| SHp268 | | pGADGH-FtsZ^{D39N,D373G,P375L} | Example 1 |
| SHp269 | | pGADGH-FtsZ^{D39N,D373G,P375S} | Example 1 |
| SHp100 | | pAS2-1-FtsA | Example 1 |
| SHp200 | | pGAD424-FtsZ | Example 1 |
| SHp201 | | pGAD424-FtsZ^{D373G} | Example 1 |
| SHp202 | | pGAD424-FtsZ^{D373S} | Example 1 |
| SHp203 | | pGAD424-FtsZ^{D373G,P375L} | Example 1 |
| | pET28 | kan^{r} lacI T7lac-(His6)tag | Novagen |
| | pBIOTRX-BirA | bla birA (biotag)trxA | (38) |
| pETbio- | | kan^{r} lacI T7lac-(biotag) | Example 1 |
| birA | | | |
| SHp173 | pDB312 | bla P_{T7}-FtsZ | Example 1 |
| LP7418 | | pUC19-FtsZ^{D39N,D373S} | Example 1 |
| pEG028 | | bla T7p-lacO-FtsZ^{D39N,D373S} | Example 1 |
| pEG045 | | pETbio-birA-ftsZ | Example 1 |
| pEG051 | | pETbio-birA-ftsZ^{D373G} | Example 1 |
| pEG052 | | pETbio-birA-ftsZ^{D373S} | Example 1 |
| pEG053 | | PETbio-birA-ftSZ^{D373G,P375L} | Example 1 |
| | pMLB1113 | bla | (14) |
| SHp185 | | pDB312-FtsZ^{D373G} | Example 1 |
| SHp187 | | pDB312-FtSZ^{D373S} | Example 1 |
| SHp189 | | pDB312-FtSZ^{D373G,P375L} | Example 1 |
| SHp174 | pDR3 | bla P_{lac}-FtsZ | Example 1 |
| SHp179 | | pDR3-FtsZ^{D373G} | Example 1 |
| SHp181 | | pDR3-FtsZ^{D373S} | Example 1 |
| SHp183 | | pDR3-FtsZ^{D373G,P375L} | Example 1 |
| SHp275 | | pGAD424-FtsZ^{D370A} | Example 1 |
| SHp276 | | pGAD424-FtsZ^{Y371A} | Example 1 |
| SHp277 | | pGAD424-FtSZ^{L372A} | Example 1 |
| SHp278 | | pGAD424-FtsZ^{F377A} | Example 1 |
| SHp279 | | pGAD424-FtsZ^{L378A} | Example 1 |
| SHp280 | | pGAD424-FtsZ^{R379A} | Example 1 |
| SHp281 | | pGAD424-FtsZ^{K380A} | Example 1 |
| SHp282 | | pGAD424-FtsZ^{Q381A} | Example 1 |

Bacteria

| Name | Genotype | Source |
|---|---|---|
| KC8 | TrpC9830 leuB600 hsdR pyrF:Tn5 | Clontech |
| | hisB463 lacDX74 strA galU galK | |
| BL21(DE3)pLysS | cam^{R} | Novagen |
| CH3 | dadR trpE trpA tna recA::Tn10 | (9) |
| PB143 | dadR trpE trpA tna ftsZ° recA::Tn10 | (30) |

Yeast

| Name | Common Name | Genotype | Source |
|---|---|---|---|
| SHy5 | EGY48 | MATα ura3-52 his3 trp1 | Clontech |
| | | GAL1p-lexA(OP)6-LEU2 | |
| SHy6 | YM4271 | MATa ura3-52 his3-200 | Clontech |
| | | lys2-801 ade2-101 ade5::hisG | |
| | | trp1-901 leu2-3,112 tyr1-501 | |
| | | gal4Δ gal80Δ | |
| SHy4 | CG1945 | Mata ura3-52 his3-200 | Clontech |
| | | ade2-101 lys2-801 trp1-901 | |
| | | leu2-3,112 gal4Δ gal80Δ | |
| | | LYS2::GAL1_{UAS}-HIS3_{TATA}- | |
| | | HIS3 | |
| | | URA3::GAL1_{UAS}-GAL1_{TATA}- | |
| | | lacZ | |
| SHy22 | | Matα ura3-52 his3-200 | Example 1 |
| | | ade2-101 lys2-801 trp1-901 | |
| | | leu2-3,112 gal4Δ gal80Δ | |
| | | LYS2::GAL1_{UAS}-HIS3_{TATA}- | |
| | | HIS3 | |
| SHy23 | | Mata ura3-52 his3-200 | Example 1 |
| | | ade2-101 lys2-801 trp1-901 | |
| | | leu2-3,112 gal4Δ gal80Δ | |
| | | LYS2::GAL1_{UAS}-HIS3_{TATA}- | |
| | | HIS3 | |
| SHy63 | | [CG1945]SHp227 | Example 1 |

TABLE 2
Oligonucleotides used in Example 1

| | | |
|---|---|---|
| FtsZ-5′ | CGCGCCAATTGATGTTTGAACCAATGGAACTTACC | (SEQ ID NO: 4) |
| FtsZ-3′ | GGCCGGGTCGACTTAATCAGCTTGCTTACGCAGG | (SEQ ID NO: 5) |
| FtsZ-5′(311) | GGGCCCGAATTCGCGACAGGTATCGGCATGG | (SEQ ID NO: 6) |
| FtsA-5′ | GGGCCCGAATTCATAACAACAATGATCAAGGGCGACGG | (SEQ ID NO: 7) |
| ZipA-5′(23) | GGGCCCGAATTCATGGTTTCTGGACCAGCCG | (SEQ ID NO: 8) |
| ZipA-3′ | GGCCGGGTCGACTCAGGCGTTGGCGTCTTTG | (SEQ ID NO: 9) |
| GALad | ATCAAAGTGGGAATATTGCTGATAGC | (SEQ ID NO: 10) |
| ADHT | GCATGCCGGTAGAGAGGTGTGG | (SEQ ID NO: 11) |
| D373G/T | GCGAAAGAGCCGGATTATCTGGGTATCCCAGCATTCCTGCGTAAGC | (SEQ ID NO: 12) |
| D373G/B | GCTTACGCAGGAATGCTGGGATACCCAGATAATCCGGCTCTTTCGC | (SEQ ID NO: 13) |
| D373S/T | GCGAAAGAGCCGGATTATCTGTCTATCCCAGCATTCCTGCGTAAGC | (SEQ ID NO: 14) |
| D373S/B | GCTTACGCAGGAATGCTGGGATAGACAGATAATCCGGCTCTTTGC | (SEQ ID NO: 15) |
| D373G, | GCGAAAGAGCCGGATTATCTGGGTATCCTGGCATTCCTGCGTAAGC | (SEQ ID NO: 16) |
| P375L/T | | |
| D373G, | GCTTACGCAGGAATGCCAGGATACCCAGATAATCCGGCTCTTTCGC | (SEQ ID NO: 17) |
| P375L/B | | |
| D370A/T | GCGCCGCAAACTGCGAAAGAGCCGGCTTATCTGGATATCCCAGCATTCCTGCGTAAGC | (SEQ ID NO: 18) |
| D370A/B | GCTTACGCAGGAATGCTGGGATATCCAGATAAGCCGGCTCTTTCGCAGTTTGCGGCGC | (SEQ ID NO: 19) |
| Y371A/T | GCGCCGCAAACTGCGAAAGAGCCGGATGCTCTGGATATCCCAGCATTCCTGCGTAAGC | (SEQ ID NO: 20) |
| Y371A/B | GCTTACGCAGGAATGCTGGGATATCCAGAGCATCCGGCTCTTTCGCAGTTTGCGGCGC | (SEQ ID NO: 21) |
| L372A/T | GCGCCGCAAACTGCGAAAGAGCCGGATTATGCGGATATCCCAGCATTCCTGCGTAAGC | (SEQ ID NO: 22) |
| L372A/B | GCTTACGCAGGAATGCTGGGATATCCGCATAATCCGGCTCTTTCGCAGTTTGCGGCGC | (SEQ ID NO: 23) |
| F377A/T | GCGAAAGAGCCGGATTATCTGGATATCCCAGCAGCCCTGCGTAAGCAAGCTGATTAAG | (SEQ ID NO: 24) |
| F377A/B | CTTAATCAGCTTGCTTACGCAGGGCTGCTGGGATATCCAGATAATCCGGCTCTTTCGC | (SEQ ID NO: 25) |
| L378A/T | GCGAAAGAGCCGGATTATCTGGATATCCCAGCATTCGCGCGTAAGCAAGCTGATTAAG | (SEQ ID NO: 26) |
| L378A/B | CTTAATCAGCTTGCTTACGCGCGAATGCTGGGATATCCAGATAATCCGGCTCTTTCGC | (SEQ ID NO: 27) |
| R379A/T | GCGAAAGAGCCGGATTATCTGGATATCCCAGCATTCCTGGCTAAGCAAGCTGATTAAGAATTG | (SEQ ID NO: 28) |
| R379A/B | CAATTCTTAATCAGCTTGCTTAGCCAGGAATGCTGGGATATCCAGATAATCCGGCTCTTTCGC | (SEQ ID NO: 29) |
| K380A/T | GCGAAAGAGCCGGATTATCTGGATATCCCAGCATTCCTGCGTGCGCAAGCTGATTAAGAATTGACTGGCGC | (SEQ ID NO: 30) |
| K380A/B | GCGCCAGTCAATTCTTAATCAGCTTGCGCACGCAGGAATGCTGGGATATCCAGATAATCCGGCTCTTTCGC | (SEQ ID NO: 31) |
| Q381A/T | GCGAAAGAGCCGGATTATCTGGATATCCCAGCATTCCTGCGTAAGGCAGCTGATTAAGAATTGACTGGCGC | (SEQ ID NO: 32) |
| Q381A/B | GCGCCAGTCAATTCTTAATCAGCTGCCTTACGCAGGAATGCTGGGATATCCAGATAATCCGGCTCTTTCGC | (SEQ ID NO: 33) |
| BIOTAG/T | CATGGCTGGTGGTCTGAACGATATCTTCGAAGCTCAGAAAATCGAATGGCA | (SEQ ID NO: 34) |
| BIOTAG/B | TATGCCATTCGATTTTCTGAGCTTCGAAGATATCGTTCAGAGCACCAGC | (SEQ ID NO: 35) |
| BirA 5′ | TATGCCATTCGATTTTCTGAGCTTCGAAGATATCGTTCAGACCACCAGC | (SEQ ID NO: 36) |
| BirA 3′ | GCCACGACCTCGAGTTATTTTTCAGCACTACGCAGGG | (SEQ ID NO: 37) |

3. Results

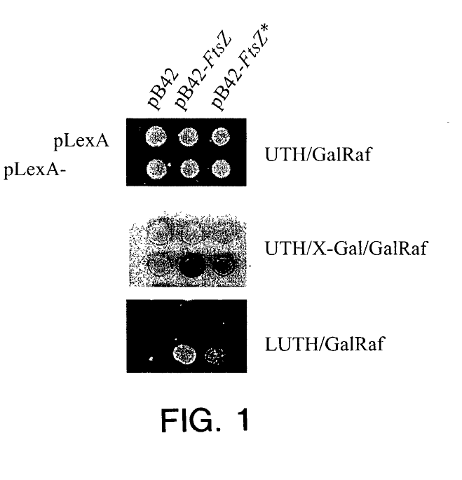

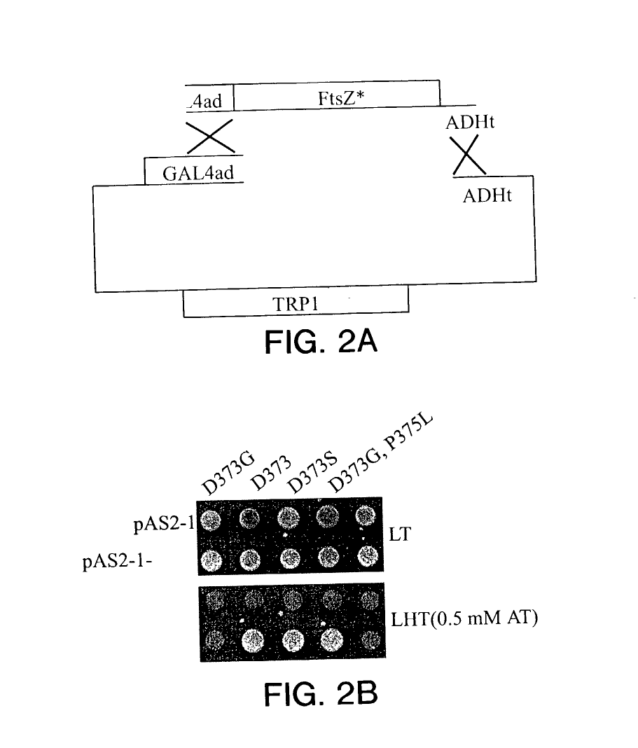

A two-hybrid system that allows for the selection of mutations in FtsZ which affect its interaction with ZipA. The interest in developing a genetic system for the characterization of the FtsZ-ZipA interaction was piqued by the observation that a mutation in FtsZ resulted in a reduced interaction with ZipA. This observation was made when one of several clones derived by PCR amplification of E. coli genomic DNA was subcloned into the two standard two-hybrid systems. When FtSZ^{N39D, D373G} (SHp48) was expressed as a hybrid to the B42 activation domain, an interaction was seen with ZipA fused to the LexA DNA binding domain. This interaction could clearly be scored in standard analyses, such as indicator plates containing X-Gal and leucine prototrophy, but was significantly less robust than another clone (SHp256) which did not contain any mutations (FIG. 1). No interaction was seen when the mutated FtsZ allele was examined in the Gal system, despite strong growth of the wild type interaction (FIG. 2B, and results not shown).

An examination of the mutations introduced during cloning suggested that one of them could be important for the FtsZ-ZipA interaction. The first mutation encoded the change N39D, which was determined by Wang et al. (40) to be one that affects the GTPase activity of FtsZ. This change was discounted as the cause of the altered interaction with ZipA by several experiments (results not shown). This evidence includes the observation that ZipA binding is not affected by guanine nucleotides, and that deletion analysis of FtsZ had already shown that the GTPase domain of FtsZ was not involved in binding to ZipA. The other mutation, D373G, resulted in a change in a highly-conserved region of the C-terminus of FtsZ (discussed below).

It is not surprising that some of the clones obtained from PCR amplification of the FtsZ gene resulted in a selection for mutations in FtsZ. Escherichia coli is very sensitive to the expression level of many of the cell division genes. Cloning of such genes on high-copy plasmids often selects for mutations in these genes, since such mutations compensate for the increased expression level. Thus, the inventors studied how a mutation with diminished function could be used to characterize FtsZ-ZipA interaction.

The lack of growth in the Gal system provided a clear strategy for determining, through the selection of intragenic suppressors, which residues in FtsZ facilitate binding to ZipA. Selection of suppressors that restored the FtsZ-ZipA interaction was achieved by PCR mutagenesis of the whole FtsZ gene, as cloned into pGAD424, using primers that annealed to the GAL4 activation domain and the ADH terminator. The primers allowed approximately 300-bp extensions to both ends of the FtsZ gene. These extensions provided regions of homology that allowed the PCR products to be cloned by recombination (FIG. 2A) (29). The PCR products were transformed directly into strain SHy63 using the pGADGH vector; the vector had been linearized, and was, therefore, not stable until it was repaired. Repair was achieved by homologous recombination with the ends of the PCR products—containing portions of the GAL4 activation domain gene—and the ADHt terminator.

Strain SHy63 contained pAS2-1-ZipA (SHp227), which allowed selection of recombinant plasmids that interacted with ZipA and activated the GAL1p-HIS3 reporter. Seventy-three colonies grew on plates which lacked histidine and which were supplemented with 1 mM aminotriazole, two of which are shown in FIG. 2B ((R373S (SHp264) and D373G, P375L (SHp267)). Plasmids were recovered and retested in yeast. Twelve purified plasmids were recovered; these had normal restriction analysis patterns, and conferred plasmid-dependent phenotypes (and histidine prototrophy only when introduced into a strain that carried SHp227). The FtsZ genes in these 12 plasmids were sequenced to identify any mutations. Eight of the 12 plasmids contained mutations in the conserved C-terminus region. The remaining four plasmids did not contain mutations that resulted in amino acid changes in FtsZ. These presumably contained mutations that affected the copy number of the plasmid or the expression of the Gal4-FtsZ hybrid protein, and so have not been further characterized.

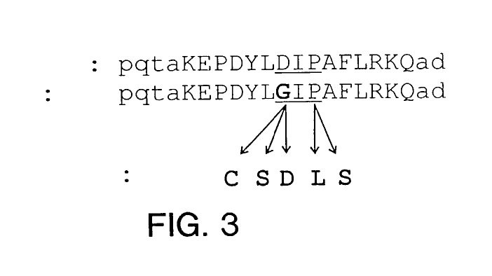

The eight mutations in the C-terminus region identified five residue changes from the original plasmid. Two mutations were reversions to the wild type aspartate residue at position 373; two mutations changed the G373 residue to serine; and the final mutation changed the glycine to cysteine. The remaining suppressors changed the highly-conserved proline residue at position 375 to leucine (twice) and to serine. These mutations are summarized in FIG. 3. The residues that comprise the C-terminus of the E. coli FtsZ are shown. Residues in capital letters tend to show some conservation among FtsZ genes from prokaryotes and plants, and some are highly conserved. The mutations identified in yeast as being critical for the interaction of FtsZ with ZipA map within this sequence; no mutations from other regions of FtsZ were identified, thereby suggesting that these residues comprise the sole region of interaction with ZipA.

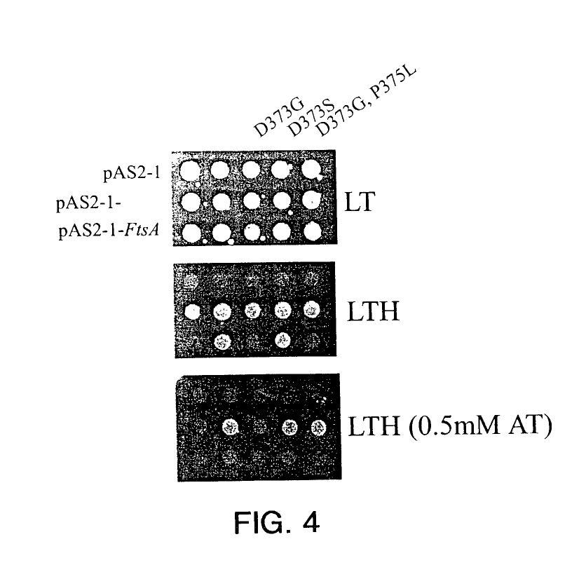

The suggestion that these mutations have significant effects on the interaction of FtsZ with ZipA was confirmed by introducing some of them into a non-mutated, wild type pGAD424-FtsZ plasmid (SHp200), and reassessing the phenotypes. Wild type FtsZ was compared to FtSZ^{D373G}, FtsZ^{D373S}, and FtsZ^{373G, P375L}. The inventors checked the interaction between FtsZ and FtsA using the two-hybrid system (FIG. 4). Because other work has suggested that FtsA interacts with FtsZ at its C-terminus, the inventors were also interested in characterizing this interaction (22, 40).

As can be seen in FIG. 4, the FtsZ-FtsA interaction is also highly sensitive to mutations in the C-terminus of FtsZ. All strains tested show good growth on plates supplemented with histidine (FIG. 4, top panel). The interaction of ZipA with FtsZ is best scored on plates containing 0.5 mM AT (FIG. 4, bottom panel), where robust growth is seen for the wild type FtsZ, and for the two suppressors identified in FIG. 2. The Gal4-ZipA fusion has a higher background than either the vector control or the Gal4-FtsA fusion, giving growth on plates without AT, regardless of which Gal4_{ad} construct is expressed (FIG. 4, middle panel). In the case of FtsA, the interaction is more sensitive, and its interactions with FtsZ can be scored on plates lacking histidine and AT (FIG. 4, middle panel). In this case, FtsA interacts well with wild type FtsZ, and with the FtsZ^{D373S} allele. It does not interact with either allele that changes the D373 residue to glycine.

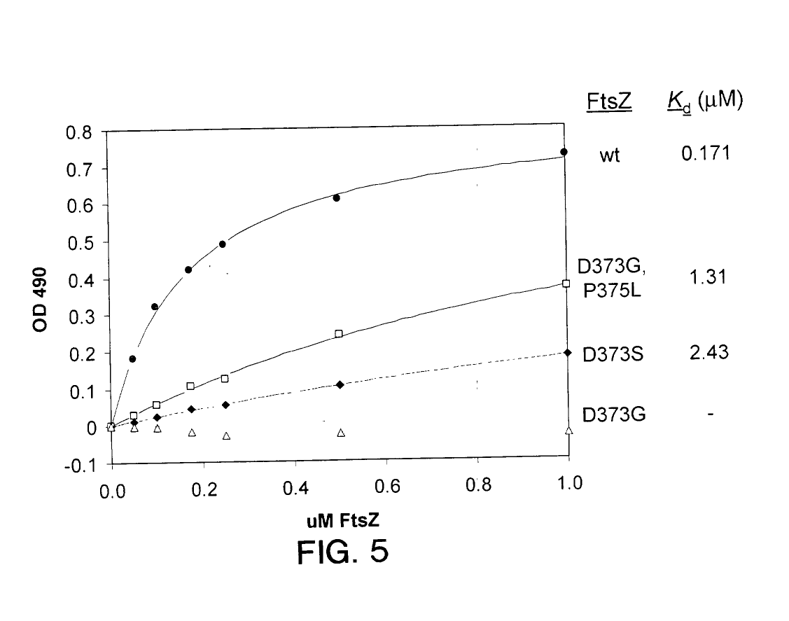

The impact of these changes on the FtsZ-ZipA interaction was also tested in vitro. In this experiment, E. Coli ZipA(23-328) and FtsZ proteins, expressed from the alleles examined in FIG. 3, were purified to homogeneity, and the interactions were tested in an ELISA. The results in FIG. 5 provide support for the genetic analysis. Wild type FtsZ shows a high affinity for ZipA, having an apparent dissociation constant of 0.17 μM. The affinity for ZipA of the FtsZ^{D373G} mutant could not be determined in this assay, despite the fact that the protein was otherwise well-behaved, thereby indicating that the mutation had a significant impact on the interaction of these two proteins. Two suppressors were analyzed as well, FtsZ^{D373S} and FtsZ^{D373G, P375L}, and these were shown to have dissociation constants of 2.4 and 1.3 μM, respectively. Both proteins exhibited greatly improved interactions with ZipA, although neither protein was wild type in its interactions with ZipA. Thus, the mutations identified by genetic analysis in yeast have indeed identified residues that are critical for the interaction of FtsZ with ZipA.

FtsA also binds to the conserved C-terminus of FtsZ, but in a manner different from ZipA. The results in FIG. 4, as well as work by others (5, 18, 22), strongly implicates the C-terminus of FtsZ as playing a critical role in the interaction of two essential components of the cell division machinery in E. coli: ZipA and FtsA. However, FtsA and ZipA show no detectable homology to each other. The peptide binding region of ZipA is structurally similar to the RNA binding site of many RNA-binding proteins (27, 28), but it is certainly distinct from the ATPase family, within which FtsA resides.

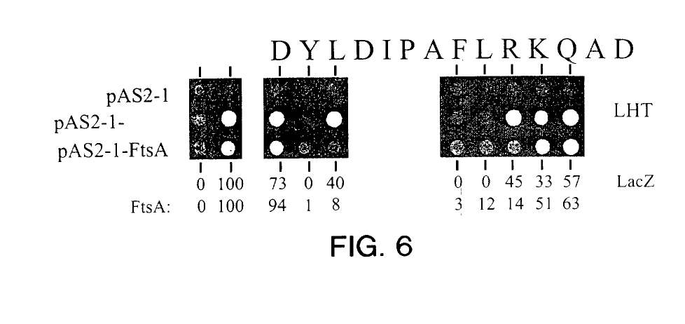

The inventors sought to determine whether ZipA and FtsA interact with identical residues in the FtsZ C-terminus. Eight additional residues within this conserved sequence were mutated individually to alanine. The residues changed to alanine are indicated in FIG. 6. These comprise additional highly-conserved residues in the FtsZ C-terminus. The figure indicates which strains expressing derivatives of pGAD424-FtsZ had specific residues changed to alanine. The strains were characterized for histidine prototrophy and for β-galactosidase activity. The figure shows that clear differences exist between the manners in which ZipA and FtsA interact with the C-terminus of FtsZ. The results of this study are generally consistent with an in vitro analysis of the interaction of a fragment of ZipA that has been shown to bind the conserved FtsZ C-terminus. Key residues for the interaction included Y371, L372, F377, and L378. The D373 residue, identified as one that affected binding when changed to glycine, showed good interaction with ZipA when changed to alanine (27), serine (FIGS. 2, 4, and 5), and cysteine.

In contrast, several changes affected the interaction with FtsA to a greater extent than the interaction with ZipA. These included L372, F377, L378, and R379. The effect of Y371A on the interaction with FtsA seemed less severe than its effect on the interaction with ZipA. The severe effect of the R379A change was interesting because structural data showed that the arginine residue was solvent-exposed and pointed away from ZipA, thereby supporting the modest effect which the change had on the interaction with ZipA in yeast. In contrast, this change had a significant effect on the interaction with FtsA, indicating that the arginine residue was important for this interaction. From this data, it can be concluded that residues in the FtsZ C-terminus play a critical role in the interaction with FtsA; however, the key residues in this interaction differ from those which play a critical role in the interaction with ZipA.

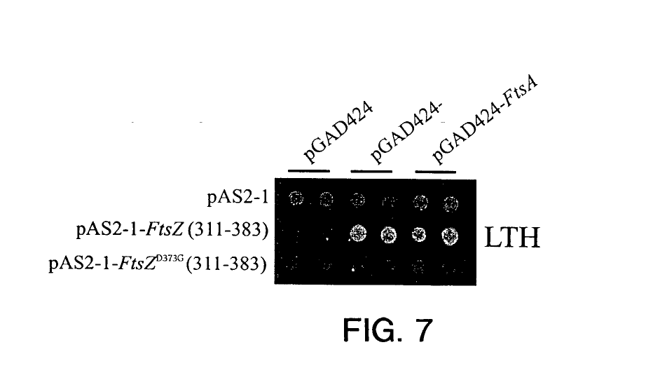

The residues which the conserved C-terminus of FtsZ requires for its interactions with ZipA and FtsA were further tested in yeast (FIG. 7). A short region of the FtsZ gene that excluded the tubulin-like core of FtsZ still interacted strongly with both ZipA and FtsA in a two-hybrid system. A similar construct that encoded the D373G variant did not interact with either fusion. Thus, the conserved C-terminus of FtsZ is both necessary and sufficient for its interaction with ZipA and FtsA.

Phenotypic consequences of mutations that change residues in the FtsZ C-terminus. The mutations described above were assayed in E. coli to determine if there were any biological consequences associated with them. The mutations were introduced into pDR3, a plasmid which can express FtsZ under a lac promoter and complement an ftsz deletion when induced by IPTG. Two assays were performed. In the first, the ftsZ alleles were characterized for their ability to complement an ftsZ deletion. The results are presented in Table 3. This experiment assessed the ability of pDR3, and of plasmids derived therefrom (see, Table 1), to complement a depletion of wild type FtsZ. pDR3 efficiently complemented in the presence of IPTG, whereas none of the mutants did. Additional dominant effects can be seen in Table 4, which examines the expression of FtsZ from the same plasmids as those in Table 3. Expression of extrachromosomal FtsZ was toxic at high levels, as was seen when a strain carrying pDR3, that was exposed to high concentrations of IPTG, expressed the plasmid copy of the FtsZ gene to high levels.

Dominant effects were observed for all of the mutated alleles, although particularly strong effects were seen with the FtSZ^{D373G} allele (SHp179) and the FtsZ^{D373G, P375L} allele (SHp183). In these cases, a Sep⁻ phenotype was observed at substantially lower concentrations of IPTG. The same was true for the FtsZ^{D373S} allele (SHp181), which had a more modest phenotype, as compared with the other mutations in this assay, but which was still substantially more toxic than the wild type gene expressed on a plasmid. Thus, a comparison between the ftsZ alleles and the wild type FtsZ indicates that all of the mutations examined in Tables 3 and 4 showed profound defects in cell division.

TABLE 3
Complementation of a FtsZ deletion by alleles
of FtsZ expressed from a regulated promoter

| | Temp (° C.)/IPTG (mM)) | | | |
|---|---|---|---|---|
| Plasmid | 30/0 | 42/0 | 42/5 | 42/10 |
| pMLB1113 | 787 | 0 | 0 | 0 |
| pDR3 | 729 | 8 | 554 | 516 |
| SHp179 | 642 | 0 | 0 | 0 |
| SHp181 | 605 | 0 | 0 | 0 |
| SHp183 | 550 | 0 | 0 | 0 |
| Plasmids were transformed to strain PB143/pCX41 [dadR- trpE- trpA- tna-recA::Tn10 ftsZ°/repAts ftsZ+], and transformants were grown overnight at 30° C. in LB + Ap (50 mg/ml) + Cam (50 mg/ml) + glucose (0.1%). Cultures were diluted in LB to OD600 = 1.10-7, and 0.1 ml aliquots were spread on four plates. One plate containing LB + Ap (50 mg/ml) + glucose (0.2%) was incubated at 30° C. The other plates were incubated at 42° C. These | | | | |
| # contained LB + Ap (50 mg/ml) supplemented with no, 5, or 10 mM IPTG. For each plate, the number of colony-forming units was determined. | | | | |

TABLE 4
Dominant effects of FtsZ mutations expressed in E. coli

| | [IPTG] mM | | | | |
|---|---|---|---|---|---|
| Plasmid | 0 | 25 | 50 | 100 | 250 |
| pMLB1113 | WT | WT | WT | WT | WT |
| pDR3 | WT/Min- | WT/Min- | WT/Min- | Min-/Sep- | Sep- |
| SHp179 | WT/Min- | Min-/Sep- | Sep- | Sep- | Sep- |
| SHp181 | WT/Min- | WT/Min- | WT/Min- | Sep- | Sep- |
| SHp183 | WT/Min- | Sep- | Sep- | Sep- | Sep- |
| Plasmids were transformed to strain CH3 [dadR- trpE- trpA- tna-recA::Tn10], and transformants were grown overnight at 37° C. in LB + Ap (50 mg/ml) + glucose (0.1%). Cultures were diluted 200-fold in LB + Ap (50 mg/ml), supplemented with the indicated concentration of IPTG, and growth was continued at 37° C. for 4-5 hr until OD600 = 0.8-1.0. Division phenotypes were determined using phase microscopy. WT: wild type; WT/Min-: | | | | | |
| #mixture of short rods and minicells - typical FtsZ-overexpression phenotype; Min-/Sep-: mixture of minicells, normal rods, and filaments; Sep-: long filaments, with very few division septa present. | | | | | |

4. Discussion

The C-terminus of FtsZ is necessary and sufficient for interaction with ZipA.

The foregoing work demonstrates that a short, conserved, region in the C-terminus of FtsZ is critical for interaction with ZipA. In making this determination, one important consideration was the fact that a search for suppressors of a loss-of-function allele identified only residues in this conserved sequence, even though the entire FtsZ gene was subjected to mutagenic PCR. This strongly suggests that this C-terminus region is the critical segment of FtsZ required for ZipA interaction.

When these results were first obtained, it was surprising that second-site intragenic suppressors were found at only one residue, P375. It appears that mutations which increase the affinity of ZipA for the D373G mutant of FtsZ could be restricted to the conserved residues of the FtsZ C-terminus, if this is the principal site of interaction with ZipA. However, the inventors were puzzled by the existence of suppressors at a single residue. The effect which these residues have on FtsZ-ZipA interaction has since been explained by structural analyses (27, 28). While the D373 residue plays a critical role in the interaction between FtsZ and ZipA, it is not directly involved in the interaction with ZipA per se. Rather, it plays a critical role in defining the structure of the residues that significantly contact ZipA. The P375 residue plays a similar structural role in defining the ZipA binding site. The residues that interact with ZipA directly (D370, Y371, L372, I374, F377, L378, and Q381) surround the D373 and P375 residues (27); therefore, the structural perturbation induced by the D373G change is alleviated by the P375L mutation.

Two other factors probably help to explain the results which the inventors obtained. First, suppressors in a heterologous system limit the types of mutations that may be recovered. In a functional screen, such as a screen for suppressors of a defect in E. coli cell division, intragenic suppressors in FtsZ may allow a bypass of the role played by ZipA. Since it is the role of ZipA to alter the dynamics of Z-ring assembly, mutations that alter GTPase activity or FtsZ-FtsZ interactions could score in a screen for functional suppressors. Intragenic suppressors in a heterologous system, such as the yeast two-hybrid system, would be limited to those that directly affect the interaction of FtsZ with ZipA. Second, a screen for increased interaction between FtsZ and ZipA would have fewer possible changes than a screen for decreased interactions. Since contact between these two proteins seems to be limited to a very few residues of FtsZ (most of the interaction energy being derived from three residues (27)), the changes that increase the affinity of FtsZ^{D373G} for ZipA may be limited by those changes which impinge on the orientation of the strongly-interacting residues.

The conserved sequence that is the focus of the experiments in this Example has also been the subject of several other earlier studies. In particular, two additional studies have examined this sequence by alanine-scanning. Mosyak et al. (27) studied the interaction of this sequence with ZipA in vitro, while Ma and Margolin (22) examined this sequence in vivo (for its interactions with FtsA and ZipA) and in yeast (for its interaction with FtsA).

In the in vitro system, a biosensor (Biacore) was used to study the interaction of the wild type peptide with ZipA. The interaction of the peptide with ZipA was weaker than that which was seen for the full length FtsZ (FIG. 4). If the interaction between ZipA and the C-terminus of FtsZ were really 20 μM, then it would suggest that other regions of FtsZ play additional, and possibly major, roles in binding. However, this would also suggest that missense mutations within this conserved sequence should have minor effects on the interaction of FtsZ with ZipA. In fact, the relative effects of the alanine-scanning mutations measured by the biosensor and in yeast indicate that the residues characterized in these studies do, in fact, play the roles suggested by the structural studies (27, 28). The results in FIG. 7 demonstrate that any other residues of FtsZ that interact with ZipA and FtsA are limited to the non-globular region at the C-terminus. The portion of FtsZ encoded by Y2H fusion plasmid in this figure consists of the last 72 residues of FtsZ, including the two β-sheets that define the beginning of the region of FtsZ that is highly divergent from the tubulins (20, 32). At present, a role for this additional sequence has not yet been established, either through the interaction of additional residues, or by providing a context for the structure of the interacting residues within the conserved sequence.

In the in vivo studies, an interaction was observed between ZipA and the FtsZ^{P375A} mutant, and between ZipA and the FtsZ^{D373A} mutant—but not between ZipA and the FtsZ^{I374A} mutant. All of these observations are consistent with the results of other studies. However, an interaction was also seen with the FtsZ^{L378A} mutant—a change seen as critical in these other studies. This may indicate that the FtsZ^{L378A} mutant may be capable of suppression by conditions in vivo, such as protein concentration within the cell, or even specifically at the septum. The reasons for this distinction may provide additional insights into the ZipA-FtsZ interaction. Regarding FtsZ-FtsA interaction, the results presented here show some additional differences; however, these most likely result from measurements of subtle interactions in similar, but not identical, heterologous systems, rather than from true discordance. This is also apparent in FIG. 6, where interactions yielding similar results in the histidine prototrophy test showed different results when β-galactosidase activity was measured (FtsZ^{D370A} and FtsZ^{Y371A}). Such results were seen consistently throughout this study.

Cell division is very sensitive to changes in the interaction between FtsZ and other proteins. The suppressors identified in this Example provide information about the sensitivity of the cell division machinery to changes in the interactions of its components. The FtsZ^{D373G} allele had profound effects on cell division and viability, and conferred on the mutant protein greatly reduced affinities for ZipA and FtsA. Such results are consistent with FtsZ interaction being an important essential function of ZipA and FtsA. The suppressors confirm that these interactions are extremely sensitive. Neither of the alleles characterized in detail in this Example (FtsZ^{D373G, P375L} and FtsZ^{D373S}) complemented a deletion. In the case of the double mutation, this is less informative about the relation between affinity for ZipA and function: because it shows a dramatically-reduced affinity for FtsA, it could explain why the double mutation fails to complement. The other allele, FtsZ^{D373S}, caused more modest changes in the interactions with FtsA and with ZipA. No change in FtsA interaction was seen in the yeast two-hybrid assay, indicating that any change in FtsA's interaction with FtsZ is relatively minor. For ZipA, an in vitro analysis showed that the protein interacts with FtsZ^{D373S} almost as well as with wild type FtsZ (FIG. 5). Although it is possible that another protein also interacts with FtsZ at this site, the simplest conclusion to draw at this time is that the proteins that function in cell division cannot tolerate even moderate changes in their interactions.

The FtsZ-ZipA interaction is a good protein-protein interaction target for the development of antibacterial compounds. There are several key questions concerning the genetic and biochemical analysis presented above. The most important considers whether the mutations described above are biologically relevant. The development of the yeast two-hybrid system as a surrogate system for the genetic analysis of the interaction between FtsZ and ZipA is meaningful only if the mutations identified are functionally important. The inventors sought to address this point by characterizing the mutations in vivo, and by observing that these mutations fail to complement and have dominant effects. This is true for the suppressors as well as the FtsZ^{D373G} allele, even though the suppressors restore the interaction with ZipA to a significant extent. An additional criterion is required if the results are to describe an important antibacterial target. In such a case, it is necessary to show that the interaction between ZipA and FtsZ is not just essential, but is very sensitive to interference.

While it is true that many protein-protein interactions are essential, they cannot be identified as promising targets for the development of antibacterial agents unless the interaction is sensitive to interference. Several genes have been characterized that are functional when expressed, or have activity, at 1% of their wild type levels, as is generally seen with nonsense mutations whose phenotypes can be alleviated by informational suppressors (25, 37). In other cases, inhibition of expression or activity by 50% can be lethal. The strength of a protein-protein interaction, or of an activity, as a pharmaceutical target can be evaluated by such data. Specifically, reducing a protein-protein interaction moderately (50-80%) should have strong phenotypic consequences if it is to be regarded as a viable pharmaceutical target, because a significantly smaller proportion of the target in the cell would have to be interfered with to see the desired effect. One of the reasons that cell division is considered an important area of antibacterial research is that many of its steps are very tightly controlled, and are very sensitive to changes in expression levels. Cell division is sensitive to changes of two to four fold in the expression of FtsZ, ZipA, and other genes. If the FtsZ-ZipA interaction itself is as sensitive, then this interaction represents a potential drug target. The results presented here support that this is the case.

Yeast two-hybrid as a system for genetic analysis of protein-protein interactions. It is clear that the yeast two-hybrid system is a powerful system for the study of protein-protein interactions (2, 7). To date, most of the effort in developing this strength has been directed to improving its utility in the screening of libraries to uncover new interactions (39). However, additional effort has gone into developing methods that allow for a better characterization of an interaction of interest (10, 17, 36). In this Example, the inventors have taken advantage of commonly used techniques for genetic analysis in yeast, and applied them to a protein-protein interaction of E. coli. This has allowed the inventors not only to determine, at the genetic level, the region of FtsZ that interacts with ZipA, but has also allowed them to identify key residues in this interaction.

The need for such methods rests on the observation that most important cellular processes are as dependent on protein-protein interactions as on enzymological functions. This is true for both prokaryotic and eukaryotic systems, and, consequently, for all areas of pharmaceutical research. While inhibition of enzyme function is well understood as a means of developing therapeutics, the development of compounds that function through the inhibition of protein-protein interactions is much less well understood (3). Two clear examples of drugs which inhibit a protein-protein interaction are FK506, which inhibits the interaction of Type I TGF-β receptors with FKBP12 (16, 41), and the peptidomimetic compound BILD 1263, which inhibits the interaction of the HSV RNR subunits (19). The polymerization of tubulin by Taxol™ is another interaction affected by a chemotherapeutic; however, in this case, Taxol™ functions by stabilizing the tubulin polymer (32, 35). Protein-protein interactions may comprise large surface areas; those that do would be much less favorable as targets for drug development. Applying the yeast two-hybrid system as a vehicle for yeast genetic analysis provides a fairly rapid and general method for determining the nature of a protein-protein interaction.

### CITED REFERENCES

1. Adams et al., Methods in Yeast Genetics (Cold Spring Harbor, N.Y.: Cold Spring Harbor Press, 1997).

2. Brent and Finley, Understanding Gene and Allele Function with Two-Hybrid Methods. Annual Review of Genetics, 31:663-704, 1997.

3. Cochran, A. G., Antagonists of protein-protein interactions. Chemistry and Biology, 7:R85-R94, 2000.

4. Dai et al., Mutations in ftsZ that Confer Resistance to SulA Affect the Interaction of FtsZ with GTP. Journal of Bacteriology, 175(1):130-36, 1994.

5. Din et al., Dominant C-Terminal Deletions of FtsZ that Affect its Ability to Localize in Caulobacter and its Interaction with FtsA. Molecular Microbiology, 27(5):1051-63, 1998.

6. Erickson, H. P., Dynamin and FtsZ: Missing Links in Mitochondrial and Bacterial Division. J. Cell Biol., 148:1103-05, 2000.

7. Fields and Song, A Novel Genetic System to Detect Protein-Protein Interactions. Nature, 340:245-46, 1989.

8. Gill and von Hippel, Calculation of Protein Extinction Coefficients From Amino Acid Sequence Data. Analytical Biochemistry, 182:319-26, 1989.

9. Golemis and Brent, Searching for interacting proteins with the two-hybrid system III. In P. A. Bartel and S. Fields (eds.), The Yeast Two-Hybrid System (Oxford, UK: Oxford University Press, 1997).

10. Grossel et al., A Yeast Two-Hybrid System for Discerning Differential Interactions Using Multiple Baits. Nature Biotechnology, 17:1232-33, 1999.

11. Gutherie and Fink, Guide to Yeast Genetics and Molecular Biology, vol. 194 (San Diego, Calif.: Academic Press, 1991).

12. Hale and De Boer, Direct Binding of FtsZ to ZipA, an Essential Component of the Septal Ring Structure That Mediates Cell Division in E. coli. Cell, 88:176-85, 1997.

13. Hale and De Boer, Recruitment of ZipA to the Septal Ring of Escherichia coli is Dependent on FtsZ and Independent of FtsA. Journal of Bacteriology, 181(1):167-76, 1999.

14. De Boer et al., A division inhibitor and a topological specificity factor coded for by the minicell locus determine proper placement of the division septum in E. coli. Cell, 56: 641-649, 1989.

15. Huang et al., Interaction Between FtsZ and Inhibitors of Cell Division. Journal of Bacteriology, 178(17):5080-85, 1996.

16. Huse et al., Crystal structure of the cytoplasmic domain of the Type I TGF-β receptor in complex with FKBP12. Cell, 96:425-36, 1999.

17. Inouye et al., Genetics, 147:479-92, 1997.

18. Liu et al., Recruitment of ZipA to the Division Site by Interaction with FtsZ. Molecular Microbiology, 31(6) :1853-61, 1999.

19. Liuzzi et al., A potent peptidomimetic inhibitor of HSV ribonucleotide reductase with antiviral activity. Nature, 372:695-98, 1994.

20. Lowe and Amos, Crystal structure of the bacterial cell-division protein FtsZ. Nature, 391:203-06, 1998.

21. Ma et al., Colocalization of Cell Division Proteins FtsZ and FtsA to Cytoskeletal Structures in Living Escherichia coli Cells by Using Green Fluorescent Protein. Proceedings of the National Academy of Sciences, 93:12998-13003, 1997.

22. Ma and Margolin, Genetic and Functional Analyses of the Conserved C-Terminal Core Domain of Escherichia coli FtsZ. Journal of Bacteriology, 181(24):7531-44, 1999.

23. Ma et al., Interactions Between Heterologous FtsA and FtsZ Proteins at the FtsZ Ring. Journal of Bacteriology, 179(21):6788-97, 1997.

24. Margolin, W., The Bacterial Cell Division Machine. ASM News, 65:137-43, 1999.

25. Michaels et al., Escherichia coli thymidylate synthase:

Amino acid substitutions by suppression of amber nonsense mutations. Proceedings of the National Academy of Sciences, 87:3957-61, 1990.

26. Miller, J. H., Experiments in Molecular Genetics (Cold Spring Harbor, N.Y.: Cold Spring Harbor, 1972).

27. Mosyak et al., The bacterial cell-division protein ZipA and its interaction with the FtsZ fragment revealed by X-ray crystallography. EMBO J., 19(3): 3179-3191, 2000.

28. Moy et al., Solution structure of ZipA, a crucial component of bacterial cell division. Nature Structural Biology (submitted for publication 2000).

29. Muhlrad et al., A Rapid Method for Localized Mutagenesis of Yeast Genes. Yeast, 8:79-82, 1992.

30. Mukherjee and Lutkenhaus, Dynamic assembly of FtsZ regulated by GTP hydrolysis. EMBO J., 17:462-69, 1998.

31. Murray and Hunt, The Cell Cycle, an Introduction (Oxford, UK: Oxford University Press, 1993).

32. Nogales et al., Structure of the alpha beta tubulin dimer by electron crystallography. Nature, 391:199-203, 1998.

33. Rockmill et al., Spore Enrichment. In C. Guthrie and G. Fink (eds.), Guide to Yeast Genetics and Molecular Biology, vol. 194. (San Diego, Calif.: Academic Press, 1991) 146-49.

34. Schatz, P. J., Use of Peptide Libraries to Map the Substrate Specificity of a Peptide-Modifying Enzyme: A 13 Residue Consensus Peptide Specifies Biotinylation in Escherichia coli. Bio/Technology, 11:1138-43, 1993.

35. Schiff et al., Promotion of microtubule assembly in vitro by taxol. Nature, 277:665-67, 1979.

36. Serebriiskii et al., A Two-Hybrid Dual Bait System to Discriminate Specificity of Protein Interactions. Journal of Biological Chemistry, 274(24):17080-907, 1999.

37. Sherman, F., Suppression in the yeast Saccharomyces cerevisiae. In J. N. Strathern, E. W. Jones, and J. R. Broach (eds.), The Molecular Biology of the Yeast Saccharomyces. Metabolism and Gene Expression (Cold Spring Harbor, N.Y.: Cold Spring Harbor Press, 1982) 463-86.

38. Smith et al., A plasmid expression system for quantitative in vivo biotinylation of thioredoxin fusion proteins in Escherichia coli. Nucleic Acids Research, 26:1414-20, 1998.

39. Walhout and Vidal, A Genetic Strategy to Eliminate Self-Activator Baits Prior to High-Throughput Yeast Two-Hybrid Screens. Genome Research, 9:1128-34, 1999.

40. Wang et al., Analysis of the Interaction of FtsZ with Itself, GTP and FtsA. Journal of Bacteriology, 179(17):5551-59, 1997.

41. Wang and Lutkenhaus, FtsZ ring: the Eubacterial Division Apparatus Conserved in Archaebacteria. Molecular Microbiology, 21:313-19, 1996.

42. Rothfield and Justice, Bacterial cell division: the cycle of the ring. Cell, 88:581-84, 1997.

43. Modern Techniques of Peptide and Amino Acid Analysis (John Wiley & Sons, 1981).

44. Bodansky, M., Principles of Peptide Synthesis (New York: Springer-Verlag New York, Inc., 1984).

All publications mentioned hereinabove are hereby incorporated in their entirety. While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A ZipA-binding site of FtsZ, comprising amino acid residues D370, Y371, L372, I374, F377, L378, and Q381 of FtsZ.

2. The ZipA-binding site of claim 2, further comprising amino acid residues D373 and P375 of FtsZ.

3. The Zip-A binding site of claim 3, further comprising amino acid residues A376, R379, and K380 of FtsZ.

4. A molecule having a ZipA-binding site, said molecule consisting of 12 to 30 amino acid residues and said ZipA-binding site including the contiguous peptide sequence DYLDIPAFLRKQ (SEQ ID NO: 3).

5. The molecule of claim 4, consisting of 12 to 24 amino acid residues.

6. The molecule of claim 4, consisting of 12 to 18 amino acid residues.

7. The molecule of claim 4, which is DYLDIPAFLRKQ (SEQ ID NO: 3).

8. The molecule of claim 4, which is a portion of FtsZ.

9. A mutant FtsZ comprising the amino acid sequence set forth in FIG. 8, in which G is substituted for D at amino acid residue 373.

10. A mutant FtsZ comprising the amino acid sequence set forth in FIG. 8, in which L is substituted for P at amino acid residue 375.

11. The mutant FtsZ of claim 10, which further comprises G substituted for D at amino acid residue 373.

12. A method for identifying an agent which interacts with FtsZ, comprising the steps of:
(a) contacting FtsZ with a candidate agent; and
(b) assessing the ability of the candidate agent to bind to FtsZ at a binding site comprising amino acid residues D370, Y371, L372, I374, F377, L378, and Q381 of FtsZ.

13. The method of claim 12, wherein the binding site further comprises amino acid residues D373 and P375 of FtsZ.

14. The method of claim 13, wherein the binding site further comprises amino acid residues A376, R379, and K380 of FtsZ.

15. The method of claim 12, wherein FtsZ is contacted with the candidate agent in the presence of ZipA.

16. An agent identified by the method of claim 12.

17. A method for identifying an agent which interacts with a molecule having a ZipA-binding site, wherein said molecule consists of 12 to 30 amino acid residues and said ZipA-binding site includes the contiguous peptide sequence DYLDIPAFLRKQ (SEQ ID NO: 3), said method comprising the steps of:
(a) contacting the molecule with a candidate agent; and
(b) assessing the ability of the candidate agent to bind to the molecule at the ZipA-binding site.

18. The method of claim 17, wherein the molecule consists of 12 to 24 amino acid residues.

19. The method of claim 17, wherein the molecule consists of 12 to 18 amino acid residues.

20. The method of claim 17, wherein the molecule is DYLDIPAFLRKQ.

21. The method of claim 17, wherein the molecule is a portion of FtsZ.

22. The method of claim 17, wherein the molecule is contacted with the candidate agent in the presence of ZipA.

23. An agent identified by the method of claim 17.
